(19) United States
(12) Patent Application Publication
(10) Pub. No.: US 2005/0277840 A1
(43) Pub. Date: Dec. 15, 2005

(54) Method and system for processing periodic physiological signals

(76) Inventor: Wen Jea Whan, La Puente, CA (US)

Correspondence Address: TROXELL LAW OFFICE PLLC, SUITE 1404, 5205 LEESBURG PIKE, FALLS CHURCH, VA, 22041, US

(21) Appl. No.: 10/855,839

(22) Filed: May 28, 2004

(51) Int. Cl. A61B 5/00; A61B 5/04

(52) U.S. Cl. 600/509

(57) ABSTRACT

A method for processing periodic physiological signals relates to digitizing and decomposing the periodic signals D of a human body into poles P and residuals S, and analyzing with the numerical method by the following formula:

$${D}^{j} = {\sum }_{i = 1}^{m} {S}_{i}^{j} {ⅇ}^{- {P}_{i}^{j}}$$

A system for processing periodic physiological signals includes: at least a sensor, a physiological signal-digitizing device, and a digitized signals processing unit and establishes a parameterized database according to P^{j}ᵢ, S^{j}ᵢ, the relationship between P^{j}ᵢ and S^{j}ᵢ and the change with respect to time or locations for evaluating the physiological or mental conditions. The method and the system are applicable to processing the periodical physiological signals, such as heartbeats, respiration and ECG.

### BACKGROUND OF THE INVENTION

1. Field of the Invention

The present invention is related to a method and a system for processing periodic physiological signals, and particularly to a method and a system for digitizing the periodic physiological signals of a human body, wherein the signals are decomposed by the numerical method into poles P and residuals S for evaluating the physiological and mental conditions of the human body. The present invention is applicable to processing the periodical physiological signals, such as heartbeat, respiration and ECG of a human body.

2. Description of the Prior Art

A healthy human body will naturally have some physiological phenomenon, e.g. heartbeat, respiration and pulses. Long ago, doctors have evaluated health conditions of a person by the symptoms of these physiological phenomenons to prevent and cure the disease. Generally, the practitioner of Chinese medicine by feeling the pulse and the doctor practicing western medicine by stethoscopes or hemadynamometers observe the change of heartbeat, pulses and respiration. In this modern time, doctors could collect these periodic signals by electronic devices for primary evaluation.

Taking heartbeat as an example, the heartbeat of a human body has the feature of regular pattern and repeat appearing. The status of these physiological signals can be measured by sonography or electrocardiography (ECG). Referring to FIG. 1, taking a standard ECG consisted of a serial wave group as an instance. Each wave group representing a heartbeat cycle includes the P-wave, QRS complex, T-wave and U-wave, wherein (1) the P-wave is generated by depolarizing the atrium is the first wave of the periodic wave group and shows the depolarization procedure of left and right atrium.

(2) The QRS complex includes three waves connected closely, wherein the Q-wave is the first downward wave, the R-wave is an upright high-tip wave after the Q-wave, and the S-wave is downward after the R-wave. In case the three waves are connected closely, they are called as QRS complex and reflect the procedure of depolarizing of left and right ventricles.

(3) The T-wave located behind the ST segment is a low and long-duration wave and is generated by ventricular repolarization.

(4) The U-wave located after the T-wave is lower and smaller and is thought to relate to afterdepolarizations, which interrupt or follow repolarization. Usually the U-wave is neglected of the ECG waveforms.

Generally ECG waveforms of normal human body have clear and complete wave change. In case the body has abnormal conditions or suffers stimulations, the ECG will show a waveform different from the normal waveforms and it can be used to evaluate the health conditions of human body. However any figures of physiological signals have to be judged by a doctor with professional training to have a result. If one can know the health conditions without evaluation by the doctor, time will be saved. Further, the physiological conditions of a person will depend on mental conditions, such as excitement, nervousness, or fear. The body will have the phenomenon of increasing heartbeat rate, sweating, and vasoconstriction. It is obvious that the mental conditions can be evaluating by observing any physiological conditions.

In view of this, the inventor provides a method and a system for processing physiological signals, which take advantage of characters of time decay and periodic performance of the periodic physiological signals to obtain a primary result by digitizing and comparing the signals. In the present invention the doctor or the medico does not need judge the signals. The present invention is applicable to preventing the diseases or abnormal mental conditions.

### SUMMARY OF THE INVENTION

The primary object of the present invention is to provide a method for processing periodic physiological signals to process the physiological signals performing periodically and to provide a result for evaluating human physiological conditions by digitization and analysis.

The secondary object of the present invention is to provide a system for processing periodic physiological signals to receive periodic physiological signals by a sensor and digitize it to provide an evaluating result by comparing a parameterized database to evaluate the physiological and mental conditions of the human body.

To achieve the above objects, a processing method according to the present invention is related to digitalizing and decomposing periodic signals D of a human body into poles P and residuals S. The P represents the decay rate of signals, and the S represents the contribution value of the P. By taking j as the serial number of signals, the D^{j} is the periodic signals with different characters, or the same periodic signals in different measuring time or locations. The D^{j}, P and S satisfy the following formula:

$${D}^{j} = {\sum }_{i = 1}^{m} {S}_{i}^{j} {ⅇ}^{- {P}_{i}^{j}}$$

Wherein m represents the maximum number of poles from the decomposed periodic signals collected from a human body, the P^{j}ᵢ represents the i^{th} pole of the j^{th} periodic signals to be decomposed, and the S^{j}ᵢ represents the contribution value or residual value of the pole. The P value is larger; the decreasing rate is faster. The human body is exerted by an external stimulation and then the periodic signals are collected and digitized. The external stimulation can generate periodic signals or change the natural periodic signals. The external stimulation can be voltage, electromagnetic waves, ultrasonic waves, heat, pressure, etc. Further calculating P^{j}ᵢ, S^{j}ᵢ, the relationship between P^{j}ᵢ and S^{j}ᵢ and the change with respect to time or space (locations) is provided to estimate the physiological or mental conditions of the human body.

A system for processing periodic physiological signals according to the present invention includes at least a sensor for sensing the periodic physiological signals of a human body and outputting the physiological signals, a physiological signal-digitizing device for receiving and digitizing the physiological signals, and a digitized signals processing unit for digitalizing and decomposing periodic signals D of a human body into poles P and residuals S. And then establishing a parameterized database according to P^{j}ᵢ, S^{j}ᵢ, the relationship between P^{j}ᵢ and S^{j}ᵢ and the change with respect to time or space (locations), and outputting an evaluating result by the parameterized database or original data value to judge the physiological or mental conditions of the human body.

The present invention will be apparent after reading the detailed description of the preferred embodiments thereof in reference to the accompanying drawings.

### BRIEF DESCRIPTION OF THE DRAWINGS

FIG. 1 is a schematic diagram of standard periodic waveforms of ECG;

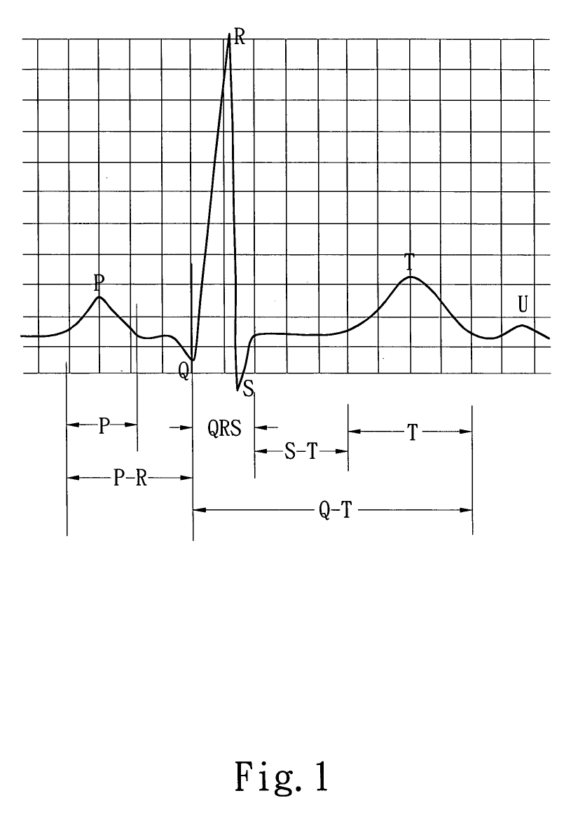

FIG. 2 is a schematic view of a system for processing periodic physiological signals of the present invention;

FIG. 3 is a periodic signals waveform diagram recorded from an ultrasonic detector measuring the heartbeat;

FIG. 4 is a periodic signals waveform diagram recorded the relative flow rate of blood vessel of thumb in different temperatures by laser Doppler flowmeter;

FIG. 5 is a three dimension waveform diagram of electromagnetic decay signals measured by eight receiving coil on the vertical position of the main blood vessel, which is exerted by external periodic electromagnetic waves; and

FIG. 6 is a schematic view of an embodiment of the present invention of the poles P^{j}ᵢ, S^{j}ᵢ and evaluating results, which are decomposed from the signals of FIG. 5.

### DETAILED DESCRIPTION OF THE PREFERRED EMBODIMENT

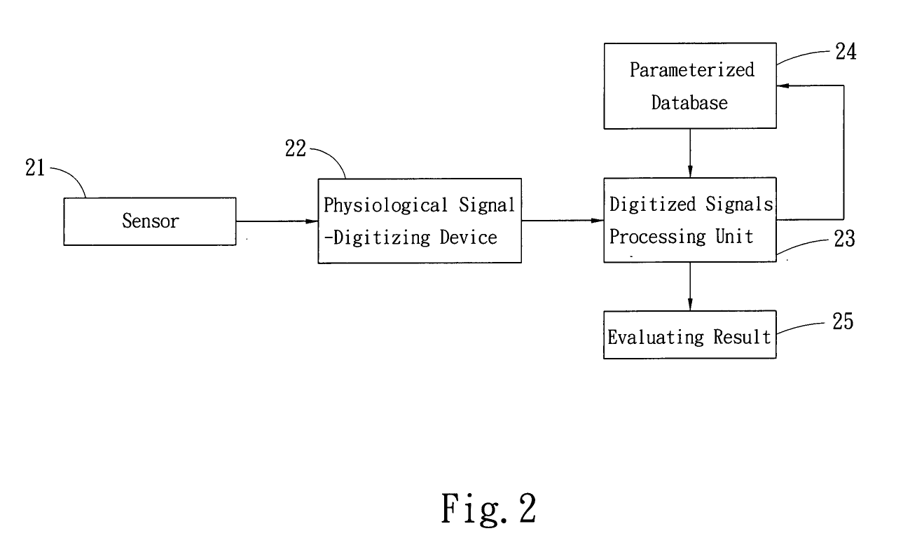

Firstly, the system for processing periodic physiological signals is depicted in FIG. 2. The system includes a sensor 21 for sensing the periodic physiological signals of a human body and outputting the physiological signals, a physiological signal-digitizing device 22 for receiving and digitizing the physiological signals, and a digitized signals processing unit 23 for digitalizing and decomposing periodic signals D of a human body into poles P and residuals S, wherein the P represents the decay rate of signals, the S represents the contribution value of the P. By taking j as the serial number of signals, the D^{j} is periodic signals with different characters, or the same periodic signals in different measuring time or locations. The D^{j}, P and S satisfy the following formula:

$${D}^{j} = {\sum }_{i = 1}^{m} {S}_{i}^{j} {ⅇ}^{- {P}_{i}^{j}}$$

Wherein m represents the maximum number of poles from the decomposed periodic signals collected from a human body. By numerical methods, P^{j}ᵢ, S^{j}ᵢ, the relationship between P^{j}ᵢ and S^{j}ᵢ and the change with respect to time or space (locations) can be established to get a parameterized database 24 for estimating the physiological or mental conditions of the human body and then output an evaluating result 25.

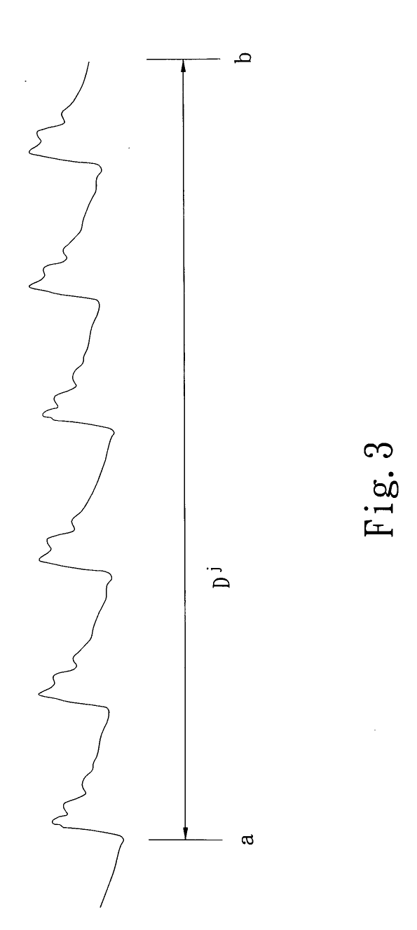

Then take a specific physiological signal as an embodiment of the present invention. Use an ultrasonic detector as the sensor 21 to measure a subject's heartbeats to obtain a heartbeat ultrasonic waveform diagram as shown in FIG. 3. Setting the range of physiological signals from point (a) to point (b) and transferring the signals between these two points to physiological signal-digitizing device 22 to obtain digitized periodic signals D. Then transferring the digitized signals to digitized signals processing unit 23 to decompose D into the poles P and the residuals S by the numerical method and the D^{j}, P and S can satisfy the following formula:

$${D}^{j} = {\sum }_{i = 1}^{m} {S}_{i}^{j} {ⅇ}^{- {P}_{i}^{j}}$$

Wherein m represents the maximum number of poles from the decomposed periodic signals collected from a human body. The P value is larger; the decreasing rate is faster. Further calculating the relationship between the P^{j}ᵢ and S^{j}ᵢ or the relationship between the P^{j}ᵢ and S^{j}ᵢ and the change with respect to time/locations and comparing those with a pre-determined parameterized database 24 to get an evaluating result 25 of the physiological or mental conditions of a human body to show the subject's health conditions.

If the parameterized database hasn't been established, following the above-mentioned method to measure enough healthy subjects in statistics and analyzing the physiological signals of the subjects to establish a parameterized database 24. Store the database 24 in the system of the present invention for the reference of evaluating the healthy conditions. The database 24 can be input be the parameterized data, e.g. gender, heights or weight, to raise the accuracy of the database 24.

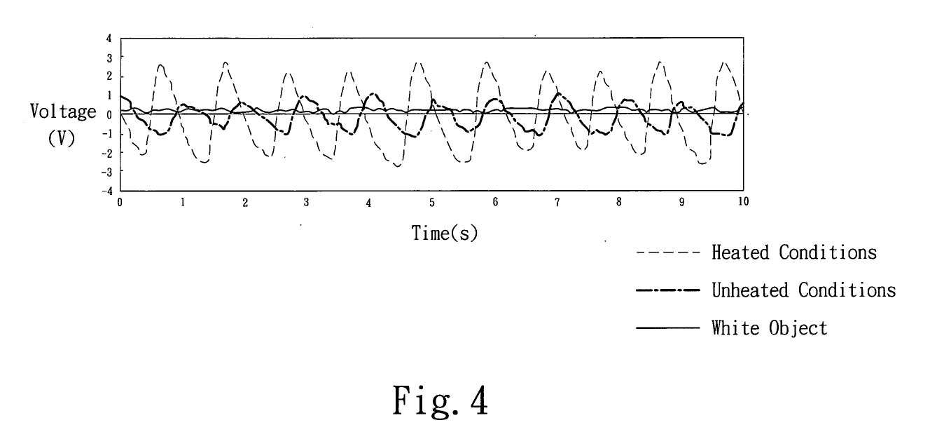

Besides, the system includes physiological signals of different measuring conditions. For example, taking laser Doppler flowmeter as a sensor 21 can measure the relative flow rate of the blood vessel of thumb in different temperatures and obtain a diagram shown in FIG. 4, wherein the control is a white object. Measuring the physiological signals in unheated conditions and in heated conditions respectively and applying the digitized signals processing unit 23 to analyze and resort the signals to further establish the physiological reference data in different measuring conditions.

The present invention is suitable for evaluating physiological signals with different characters. For example, taking the electrocardiograph as a sensor 21 of the system to detect heartbeat of a human body, and applying the system mentioned above to digitize and decompose the physiological signals to store the P^{j}ᵢ, S^{j}ᵢ and time/locations in the digitized signals processing unit 23 and compare those with the ultrasonic data of heartbeats mentioned above to further obtain the physiological and mental conditions of a human body.

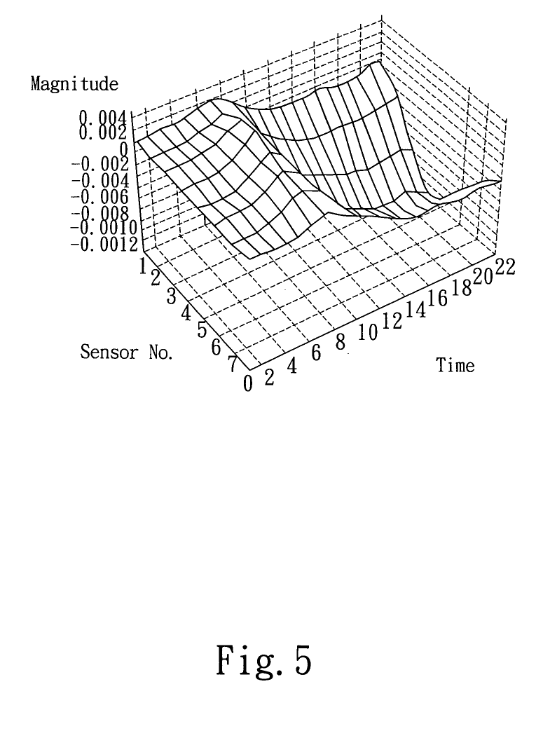

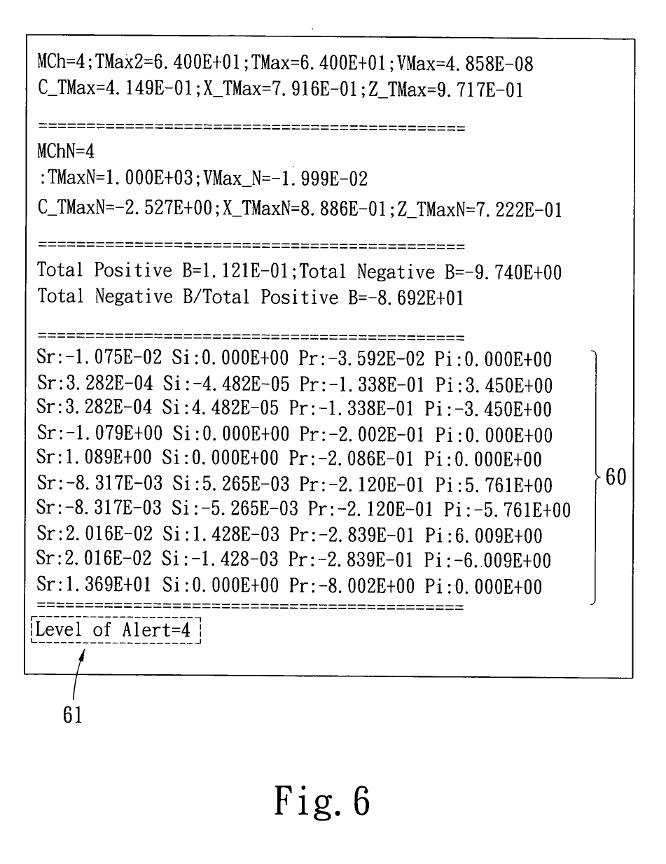

Not only for measuring the physiological signals of normal conditions, the system of the present invention can evaluate the health conditions of a human body but also by being exerted an external stimulation. The external stimulation can generate periodic signals or change the natural periodic signals. The external stimulation can be voltage, electromagnetic waves, ultrasonic waves, heat, pressure, etc. For example, by stimulating external periodic electromagnetic waves, the main blood vessel is positioned with eight receiving coils. The decay signals of electromagnetic field generated by Eddy current are shown in FIG. 5. The poles P^{j}ᵢ and residuals S^{j}ᵢ decomposed from the signals D are shown in FIG. 6 labeled as 60. Then comparing these values with the pre-determined parameterized database 24 to obtain an evaluating result of physiological or mental conditions of a human body. The evaluation result shown in FIG. 6 labeled as 61 can indicate the health level of the tested person.

The foregoing description of the preferred embodiments of this invention has been presented for purposes of illustration and description. It is not intended to be exhaustive or to limit the invention to the precise form disclosed, and obviously, many modifications and variations are possible. Such modifications and variations that may be apparent to a person skilled in the art are intended to be included within the scope of this invention.

Thus, the present invention has following advantages:
1. The present invention not only can be used as analysis of single physiological signals, but also can analyze composite physiological signals to raise the accuracy of evaluation.
2. By using the present invention to evaluate body conditions, it is controlled by electronic devices without judging by a doctor and is convenient for operating to figure out the health conditions.
3. Because the different measuring conditions will result in different conclusions, operators can input parameters with different terms, e.g. heights, weights, external stimulations or temperatures, then the parameterized database can make the results more accurately.
4. The method and system for processing periodic physiological signals not only offer the evaluation of physiological conditions but also offer the evaluation of mental conditions.

As stated in the above disclosed, the present invention can surely achieve its expected objects to provide a method and a system for processing periodic physiological signals for the public or the medical personnel to evaluate the health conditions. It has its industrial practical value. Having thus described my invention, what I claim as new and desire to be secured by Letters Patent of the United States are:

1. A method for processing periodic physiological signals, digitalizing and decomposing periodic signals D of a human body into poles P and residuals S, wherein the poles P represent the decay rate of signals, the residuals S represent the contribution value of the P, by taking j as the serial number of signals, D^{j} represent periodic signals with different characters, or the same periodic signals in different measuring time or locations, and D^{j}, P and S satisfy the following formula:
$${D}^{j} = {\sum }_{i = 1}^{m} {S}_{i}^{j} {ⅇ}^{- {P}_{i}^{j}}$$
wherein m represents the maximum number of poles from the decomposed periodic signals collected from a human body, p^{j}ᵢ represents the i^{th} pole of the j^{th} periodic human signals, S^{j}ᵢ represents the contribution value or residual value of the pole; thus, obtaining the values of the poles p^{j}ᵢ and residuals S^{j}ᵢ by decomposing the periodic signals by the numerical method.

2. The method for processing periodic physiological signals according to claim 1, wherein said human body is exerted by an external stimulation, said external stimulation can generate periodic signals or change the natural periodic signals, and then the periodic signals are collected and digitized.

3. The method for processing periodic physiological signals according to claim 2, wherein said external stimulation is voltage, electromagnetic waves, ultrasonic waves, heat or pressure.

4. The method for processing periodic physiological signals according to claim 1, wherein the value of P^{j}ᵢ or S^{j}ᵢ are used for evaluating the physiological or mental conditions of said human body.

5. The method for processing periodic physiological signals according to claim 1, further comprising calculating the relationship between the P^{j}ᵢ and S^{j}ᵢ for evaluating the physiological or mental conditions of said human body.

6. The method for processing periodic physiological signals according to claim 1, further comprising calculating the change with respect to time or locations for P^{j}ᵢ or S^{j}ᵢ for evaluating the physiological or mental conditions of said human body.

7. The method for processing periodic physiological signals according to claim 1, further comprising calculating the change with respect to time or locations for the relationship of P^{j}ᵢ and S^{j}ᵢ for evaluating the physiological or mental conditions of said human body.

8. The method for processing periodic physiological signals according to claim 1, further comprising calculating P^{j}ᵢ, S^{j}ᵢ, the relationship between P^{j}ᵢ and S^{j}ᵢ and the change with respect to time or locations to establish a parameterized database for evaluating the physiological or mental conditions of said human body.

9. A system for processing periodic physiological signals including:
at least a sensor, for sensing the periodic physiological signals of a human body and outputting said physiological signals;
a physiological signal-digitizing device, for receiving and digitizing the physiological signals; and
a digitized signals processing unit, for digitalizing and decomposing periodic signals D of a human body into poles P and residuals S, wherein the poles P represent the decay rate of signals, the residuals S represent the contribution value of the P, by taking j as the serial number of signals, D^{j} represent periodic signals with different characters, or the same periodic signals in different measuring time or locations, and D^{j}, P and S satisfy the following formula:
$${D}^{j} = {\sum }_{i = 1}^{m} {S}_{i}^{j} {ⅇ}^{- {P}_{i}^{j}}$$
wherein m represents the maximum number of poles from the decomposed collected from a human body, P^{j}ᵢ represents the i^{th} pole of the j^{th} decomposed human periodic signals, S^{j}ᵢ represents the contribution value or residual value of the pole;
thus, establishing a parameterized database according to P^{j}ᵢ, S^{j}ᵢ, the relationship between P^{j}ᵢ and S^{j}ᵢ and the change with respect to time or locations by the numerical method, and outputting an evaluating result by said parameterized database for estimating the physiological or mental conditions of said human body.

10. The system for processing periodic physiological signals according to claim 9, wherein said human body is exerted by an external stimulation, said external stimulation can generate periodic signals or change the natural periodic signals, and then the periodic signals are collected and digitized.

11. The system for processing periodic physiological signals according to claim 10, wherein said external stimulation is voltage, electromagnetic waves, ultrasonic waves, heat or pressure.

12. The system for processing periodic physiological signals according to claim 9, wherein the value of P^{j}ᵢ or S^{j}ᵢ are used for evaluating the physiological or mental conditions of said human body.

13. The system for processing periodic physiological signals according to claim 9, further comprising calculating the relationship between P^{j}ᵢ and S^{j}ᵢ for evaluating the physiological or mental conditions of said human body.

14. The system for processing periodic physiological signals according to claim 9, further comprising calculating the change with respect to time or locations for P^{j}ᵢ or S^{j}ᵢ for evaluating the physiological or mental conditions of said human body.

15. The system for processing periodic physiological signals according to claim 9, further comprising calculating the change with respect to time or locations for the relationship of P^{j}ᵢ and S^{j}ᵢ for evaluating the physiological or mental conditions of said human body.
